(19) United States
(12) Patent Application Publication
(10) Pub. No.: US 2004/0210280 A1
(43) Pub. Date: Oct. 21, 2004

(54) Plaster-type chip systems for thermodynamic control of topical dermal and transdermal systems

(75) Inventor: Rainer K. Liedtke, Gruenwald (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BIONICS PHARMA GmbH, Gruenwald (DE)

(21) Appl. No.: 10/686,586

(22) Filed: Oct. 17, 2003

(30) Foreign Application Priority Data
Oct. 25, 2002 (DE) 102 49 853.9

(51) Int. Cl. A61F 7/00; A61F 7/12; A61B 17/00

(52) U.S. Cl. 607/96; 606/1

(57) ABSTRACT

The invention pertains to patch-like chip systems for the thermodynamic control of topical dermal and transdermal systems, especially for improving the efficiency and safety of dermal and transdermal therapies and diagnoses. From an information technological standpoint, the systems represent complex technical devices that, in comparison to conventional passive systems, represent controlled and intelligent systems as a result of programmable and also individual control. Their incorporation into passive dermal or transdermal therapeutic systems requires no technical incursions into the existing structure of such systems. The patch-like chip systems also open up new usage possibilities in the sector of non-invasive and micro-invasive dermal and transdermal diagnosis.

The invention pertains to patch-like chip systems for the thermodynamic control of topical dermal and transdermal systems, and especially for improving the efficiency and safety of topical dermal and transdermal therapies and diagnoses.

It is known that the application of physical effects as well as chemical effects to, and through, the skin can permit numerous significant advantages. The applications of thermal or electrical stimuli to the surface of the skin, or to single or multiple layers of skin, and/or to tissues, which are supplemental to the skin, are examples of dermal and transdermal physical effects. In addition to their medicinally relaxing and pain alleviating effects on the neuromuscular organs and systems, mention can also be made in the case of thermal therapeutic effects of, for example, the types of application that take place by means of so-called regional hyperthermia for the treatment and sensitization of tumor tissues.

So-called transdermal systems are included among the newer therapeutic chemical applications, whereby these systems are specific technical patch systems with variously configured medicament reservoirs from which medicinal preparations are continuously released into the skin and then migrate from there into the circulation system. In exactly the same way, however, semi-solid pharmaceutical formulations also exist that contain an active substance, whereby these have already been in use for an extended period of time, e.g. ointments, gels, and creams, that are applied to the skin for resorption purposes. There are even some substances within the framework of transdermal patch systems, whereby these substances are thereby used for systemic therapy. The following, for example, are included among these: steroid hormones for hormone substitution in cases of menopausal complaints and also those for contraception; nitroglycerine in cases of angina pectoris; nicotine for breaking the habit of smoking; scopolamine in cases of travel sickness with vertigo; and the analgesic substances fentanyl and buprenorphine for the therapy of severe pain conditions. Very many formulations exist within the range of semi-solid pharmaceutical forms of medication for various usage purposes, both topically and systemically, e.g. those for alleviating local pains or neuromuscular complaints as well as formulations for locally influencing traumatic or degenerative injuries to the skin.

However, technical devices in which solid or semi-solid pharmaceutical formulations that are introduced into the tissue beneath the surface of the skin by means of invasive procedures, e.g. via implantation or injection, and that then, over extended periods of time, continuously release their active substances from this site in the form of reservoirs, are also, for example, included within the range of dermal therapeutic systems. For example, the following belong to these in the technical sense: crystal suspensions, colloidally dispersed formulations, or deposit formulations comprising biologically compatible substances that can be eroded enzymatically and that can contain e.g. analgesics or various hormones.

Technical devices, which are introduced onto, and/or into, the skin and with which information regarding bodily condition can be gained, can also be classified as dermal or transdermal diagnostic systems. For example, qualitative or quantitative information regarding the amounts of certain substances that are inherent in, or extraneous to, the body, e.g. information regarding the concentration in the blood of glucose, hormones, or electrolytes, as well as medicinal preparations or drugs, is included here.

The transfer of substances into, and through, the skin basically follows the physical principles of passive diffusion in accordance with Fick's laws of diffusion. The molecules can hereby penetrate the skin either in a trans-cellular manner, i.e. through the cells, or in an intracellular manner, i.e. via the interstitial spaces that are located between the cells. However, they can also proceed along routes via accessory skin organs, e.g. hair follicles and sweat glands. The uppermost keratin containing layer of skin, i.e. the stratum corneum, hereby constitutes a significant barrier for the majority of substances. Once diffusion through this layer of the epidermis has been achieved, the molecules readily permeate into the dermis, which is located below it, and then they are absorbed by the capillaries of the skin via which they then get into the circulation system (Karzel, K. & Liedtke, R. K.: Mechanisms of transcutaneous resorption, Arnzneim. Forsch./Drug Res. 11a (1989) 1487). Since diffusion, in a non-directed way, merely follows the concentration gradients that are in operation at that time, the same also applies to the inverse passageway, i.e. from the capillaries toward the epidermal surface where the stratum corneum likewise proves to be the main barrier.

According to Kligman (Drug Dev. Industr. Pharm. 9: 521-560, 1983), diffusion through and in the skin itself is, likewise, primarily a temperature dependent process. It is to be expected from this that a certain elevation of the skin temperature will also increase any thermodynamic driving force there. In turn, it is also to be expected from this that supplying heat will then, inter alia, also intensify the release of substances from deposits that have been introduced below the skin. For example, an increased rate of disappearance of previously injected ¹²⁵I-labelled insulin from the subcutaneous tissue was found in this connection in the case of diabetic persons following the local application of heat, whereby this was attributed to a largely linear increase in cutaneous blood flow in this regard (Hildebrandt, P. et al.: J. Clin. Lab. Invest. (1985) 45 (8) 685-690); and also: Diabetes Res. 1987, 4 (4) 179-181). The magnitude of the insulin concentration in the serum following its subcutaneous injection was also significantly statistically correlated with the skin temperature in another study involving healthy persons (Sindelka, G., et al.: Diabetologia (1994) 37 (4): 377-380).

This effect can be produced simultaneously by several physiological regulatory factors, either alone or via a combination thereof. For example: by increases in cellular skin permeability, by increases in local fluid circulation, by an increase in the permeability of the walls of blood vessels, as well as by the thermally engendered increase in chemical solubility of the substances. Investigations by Rowell et al. (J. Appl. Physiol. 28 (4) (1970) 415) showed that the cutaneous flow of blood is hereby increased at the rate of 3 L/min per ° C. increase in body temperature. External heating can induce an increase in the perfusion of blood through the skin by up to 12 times. However, locally limited heating of the skin tissue does not hereby significantly influence the core temperature of the body, but results only in a local increase in the subcutaneous flow of blood.

Various studies have been undertaken in order to show that an increase in the cutaneous flow of blood, as a result of exposure to heat, also changes the pharmacokinetics of transdermally administered substances. The results of such studies show that external heating intensifies both transdermal and subcutaneous absorption, and this then resulted in increased plasma concentrations of these substances (Vanakoski, J. et al.: Clin. Pharmacokinetics 34 (4) (1998) 311-322).

For example, the relationship between the cutaneous flow of blood and the transdermal absorption of nitroglycerine has been demonstrated in a study in which patches with nitroglycerine were placed on the upper arm. The patch area was thereby heated in an isolated manner using an infrared lamp (Klemsdal et al.: Eur. J. Clin. Pharmacol. 43 (1992) 625). Such heating intensified the local perfusion of blood and, at the same time, the concentrations of nitroglycerine in the plasma were increased by two to three times. Local cooling of the patch site with ice was again followed by a decrease in the plasma concentrations of nitroglycerine, whereby this showed that the process is reversible. In another study, Gupta et al. (J. Pain Symptom Management 7 (3) (1992) Suppl.: page 17-page 26) determined in vitro the effect of various temperatures (between 32° C. and 37° C.) on the transdermal flux of the analgesic substance fentanyl. The flux rate approximately doubled over this range of temperatures. On the basis of a pharmacokinetic model, such an increase depends mainly on two factors: the accelerated release of fentanyl from the technical reservoir of the patch together with increased skin permeability.

Thus, as is known, the aspects arise from these examples that, inter alia, transdermal pharmacodynamic effects are also capable of being triggered and intensified via the application of heat, and that the production of heat can take place via various physical means and also via chemical means, e.g. by producing exothermic chemical reactions.

An important biological mechanism for the phenomenon hereby appears to be increases, which are thermally induced in a physiological manner, in the local flow of blood in the skin as a consequence of local vascular widening, as well as local changes that result therefrom in terms of intradermal fluid circulation. In overall terms, the mechanism thus comprises permeation through the layers of skin, and diffusion between the cutaneous and subcutaneous tissue as well as that from the tissues into the circulation system. Thus the increases in the plasma concentrations of some substances that are brought about in this way indicate that, for substances that are suitable in this regard, a technically suitable device for the local application of heat can increase their release and permeation in, inter alia, a transdermal manner as well.

In contrast to a few fundamental findings that are already available, namely that the local application of heat could also promote the dermal or transdermal therapeutic use of medicinal substances, nothing is currently known in regard to the area of dermal or transdermal diagnostic procedures, i.e. in regard to dermal diagnostic procedures or devices that also depend on a local thermodynamic effect, or one that can be promoted by them.

In addition to their having suitable physicochemical properties, such as e.g. their molecular weight and solubility, the transdermal medicinal therapeutically suitable triggering of biological effects requires that the substances that are used be released in a controlled form that is also suitable for this purpose. However, this objective has not yet been achieved with previously known thermal and transdermal therapeutic systems. Thus the currently used transdermal patch systems and, likewise, semi-solid pharmaceutical formulations, merely represent purely passive diffusion systems. Thus the transportation of the substances, which are contained in them, into the circulation system depends only on the concentration difference in question between the active substance reservoir in the pharmaceutical formulation and the skin or, in the phase that follows on from here, the concentration difference between the subcutaneous tissue and the blood. This permits such devices to exhibit continuous substance release, but it in no way permits individually required changes and adaptations in regard to reproducibly controlled permeation in an individually given situation. Thus, for example, an acute increase in the dose of an analgesic in the case of a patient with pain would be required if the system does not release an adequate dose in order to effectively reduce his acute pain condition.

Thus research studies to integrate technical devices into such therapeutic transdermal systems are also known, whereby these are intended to intensify the transportation of substances through the skin, or to control in an improved manner the release of the substances from the patch system. This involves the technical use of both chemical and physical procedures.

So-called chemical enhancers form part of these chemical procedures. These are substances that are intended to make the skin permeable in an improved manner as a result of a direct chemical influence on the structure of the skin. However, the disadvantages of such substances are that they chemically destroy the biological integrity of the skin, and they are capable of producing considerable skin irritations and side effects as a result. Certain chemical agents are also known, the so-called rubefacient substances, by means of which the skin is stimulated topically and, as a result, the skin is then stimulated to give a local increase in blood perfusion in a neuronally reactive manner. Products with substances that produce such heat sensations are known in part as so-called topical “rheumatism patches”. However, the actual regulatory effects of such stimulants are controversial, and they often produce a subjective “feeling of warmth” only via local nerve stimulation, whereby this is independent of the fact that, in this regard likewise, however, one is not dealing with a controlled release mechanism. In addition, chemical procedures are also known in which chemical heat producing reactions are brought about via the release of agents that are contained in the patch itself, whereby the active substance or the patch is heated via these reactions. This procedure is also basically suitable for increasing substance permeation, but it takes place thermally in an extremely uncontrolled manner, and it also involves negative safety and tolerance aspects for the skin as a result of the use of the inorganic reagents that are required for this purpose. Thus U.S. Pat. No. 4,230,105 already pertains to a bandage with a medicinal preparation and a device that generates heat chemically. U.S. Pat. No. 4,898,592 also describes a device for using heated transdermally absorbable substances, whereby one layer here is impregnated with a transdermally absorbable substance, and another contains a thermal element. The claims of U.S. Pat. No. 4,685,911 also pertain to the application of heat via a medium, which generates heat by chemical means, in order to increase absorption. A patch with a device for the direct chemical production of heat is also described in U.S. Pat. No. 6,306,431, preferably using a mixture comprising iron powder, activated carbon, salt, and water in which atmospheric oxygen gets to the heat generating mixture after removing an airtight covering layer, whereby this subsequently brings about the triggering of an exothermic reaction. However, this mechanism for the exothermic production of heat, which is claimed as such by U.S. Pat. No. 6,306,431, is not new since U.S. Pat. No. 4,685,911 already describes exactly this form of exothermic chemical production of heat as well. Within the wider framework of their descriptions, some of these techniques also indicate general physical fields that are known as such, whereby use could be made of e.g. electrical energy for the production of heat instead of using chemical energy, and whereby electrically produced heat could also be controlled via the use of electrical devices.

Known physical procedures in the case of transdermal applications for bringing about improved control are also those by means of electricity, e.g. by means of iontophoresis, as well as by means of the use of devices involving ultrasound (in the case of which, inter alia, subcutaneous heat is also produced indirectly), and also by means of magnetic devices. At the present time, the so-called iontophoretic systems appear to be technically the farthest developed in the therapeutic transdermal sector. In the case of this technical principle, which has been known for a long time from the medical historical standpoint and which has also been used for a long time, the migration of ionized molecules takes place through an electric field that runs tangentially to the skin. The electric field is hereby produced by means of a source of electric current between two electrodes that are located separately from one another in the patch. However, these systems are technically very expensive and, in addition, relatively voluminous and unwieldy and costly as well. In addition, they are accompanied by some considerable problems in regard to tolerance by the skin, whereby this is brought about by the direct involvement of the skin as a physical supporting medium for the flow of the electric current that is produced.

The feature arises from that which has been stated above that technically isolated approaches to a solution have indeed been pursued, via individual aspects, for the therapeutic application of heat to the skin. However, an overall consideration of dermal thermodynamic processes has not become known thus far, i.e. one that describes the overall interactive mechanism and physiological effects of the topical action of heat in a technically consistent manner and that also converts this into an appropriately technical integrated and practical form. A feature that is common to all these technical devices is that they trigger their effects in an extremely uncontrolled manner since they proceed only in a unilaterally directed and irreversible way, e.g. in the case with exothermic chemical reactions that can no longer be controlled in terms of their further course. Adequate individual dosage in accordance with requirements is then not possible in this way, either. Whereas such devices for dermal and transdermal systems with individual technical process components together with those with technical process components, which have not been optimized with respect to one another in a defined way, cannot be classified as controlled systems, from the standpoint of information technology, within the therapeutic sector, there are absolutely no approaches or devices with a topical thermodynamic approach in the dermal and transdermal diagnostic sector.

The problem that forms the underlying basis of the invention is to improve the efficiency and safety of topical dermal and transdermal therapies and diagnoses.

This problem is solved by way of the feature that use is made of patch-like chip systems for the thermodynamic control of topical dermal and transdermal systems, whereby these are composed in the form of a multi-component system that is configured in a patch-like manner in such a way that they comprise a source of electrical energy, which is located in a communal supporting matrix, and a programmable microprocessor, which serves as a thermo-controller, along with an activation circuit, whereby these, for their part, are technically connected to a device that produces electrically induced heat, and whereby, in overall terms, the patch-like chip system can be applied in a complementary manner to a topical dermal or transdermal system in such a way that the heat profile that is produced is transferred to the topical dermal or transdermal systems in such a way that these [systems] are thermodynamically activated in a controlled form.

In a further form of embodiment of the invention, the communal supporting matrix is geometrically subdivided into operational function sectors in order to improve and expand practical usage, whereby the function sectors are mutually connected in an electrically conducting manner, and whereby the connections between these function sectors can be configured in a reversible manner.

In a further form of embodiment of the invention, the matrices and technically active components of the patch-like chip systems are composed of certain materials in order to improve and expand practical usage, whereby these materials possess mechanically elastic or plastic properties, and they are optically transparent or opaque, and they possess electrically conductive or magnetic properties, and, chemically, they are non-metallic polymers of natural or synthetic origin, or they are metallic materials.

In a further form of embodiment of the invention, additional electrical, electronic, magnetic, micro-mechanical, chemical or chemo-technical components, or combinations thereof, are incorporated into the devices in order to improve and expand practical usage for specific usage purposes.

In a further form of embodiment of the invention, control of the induced heat-profile takes place in order to improve and expand practical usage, whereby such control takes place either using an open-loop control technique or a closed-loop technique with feed-back via sensors.

In a further form of embodiment of the invention, devices for the reception and transmission of remote control signals are present in order to improve and expand practical usage, whereby such reception and transmission can take place either physically (via infrared, ultrasound, electromagnetic waves, or laser techniques) or in a chemosensory manner via chemically volatile substances.

In a further form of embodiment of the invention, the thermodynamic actor can also be triggered in sub-surfaces, including those with different temperatures, in order to improve and expand practical usage.

In a further form of embodiment of the invention, the thermodynamic actor is configured in the form of all possible two-dimensional geometries in order to improve and expand practical usage.

In a further form of embodiment of the invention, production takes place technically, in parts or wholly, using roll-to-roll processes in order to improve and expand practical usage.

In a further form of embodiment of the invention, the devices are used therapeutically in certain dermal and transdermal systems in order to improve and expand practical usage, whereby these systems do not contain pharmacologically active substances.

In a further form of embodiment of the invention, the devices are used therapeutically for the purpose of regional hyperthermia for locally heating tumor cells (especially those in the breast region, the skin region, or in the genital region) in order to improve and expand practical usage.

In a further form of embodiment of the invention, these [devices] are used therapeutically in certain topical dermal or transdermal systems in order to improve and expand practical usage, whereby these systems contain the following as pharmacologically active substances: nitroglycerine, fentanyl, sufentanil, buprenorphine, morphine, hydromorphine [sic; hydromorphone?], lidocaine, indomethacin, ibuprofen, diclofenac, piroxicam, nicotine, clonidine, estradiol, progesterone, testosterone, norethisterone, oxybutynin, buspirone, scopolamine, including their chemical analogs, derivatives, isomers, and salts either in the form of individual substances or in the form of combinations.

In a further form of embodiment of the invention [typo], these [devices] are used therapeutically in certain dermal or transdermal systems in order to improve and expand practical usage, whereby these systems comprise semi-solid or fluid forms as the pharmaceutical formulation such as, in particular, ointments, gels, creams, lotions, suspensions, or solutions.

In a further form of embodiment of the invention, this [device] is used for the accelerated disintegration of epidermal or dermal deposits of active substances in order to improve and expand practical usage, especially deposits containing the following hormones: insulin, growth hormone, estradiol, progesterone, and testosterone, including their chemical analogs.

In a further form of embodiment of the invention, this [device] is used in the form of a patch-like dermal diagnosis system in order to improve and expand practical usage, whereby such a diagnosis system is used for gathering and analyzing the natural fluid from the skin, sweat, and interstitial dermal fluid, and especially for the analysis of the following substances that are contained therein: glucose, lactate, electrolytes, adrenalin, creatine, medicinal preparations, alcohol, and drugs.

In a further form of embodiment of the invention, this [device] is used in the form of a patch-like non-invasive dermal or transdermal diagnosis system in order to improve and expand practical usage, whereby the collection and analysis of the fluid, which emerges onto the surface of the skin, takes place by means of collection and sensor devices, which are integrated therein, whereby the thermodynamic actor is arranged around them in a circular manner, and whereby the fluid from the skin is absorbed by a plate-like collection device, which is equipped with capillary channels, and the fluid is analyzed and evaluated by means of electronic chemosensors or chemical test strips, which are in contact with the fluid, and whereby this is used for the non-invasive analysis of, in particular, glucose, lactate, electrolytes, adrenalin, creatine, medicinal preparations, alcohol, and drugs.

In a further form of embodiment of the invention, this [device] is used in the form of a patch-like dermal or transdermal micro-invasive diagnosis system in order to improve and expand practical usage, whereby the collection and analysis of interstitial fluid from the skin takes place by means of an integrated collection and sensor device, and whereby the thermodynamic actor is arranged around it in a circular manner, and whereby the interstitial fluid from the skin is absorbed or contacted by a plate-like collection device, which is equipped with micro-tubes, and whereby this collection device is suitable for penetrating the uppermost epidermal layer of skin, and the fluid is analyzed and evaluated by means of electronic chemosensors or chemical test strips, which are in contact with the fluid, and whereby this is used for the micro-invasive analysis of, in particular, glucose, lactate, electrolytes, adrenalin, creatine, medicinal preparations, and drugs.

In a further form of embodiment of the invention, the collection and conveying device for fluid from the skin comprises, wholly or in parts, hollow polymeric fibers, micro-tubes, or hollow probes, which are made from a metallic, polymeric, or ceramic material, in order to improve and expand practical usage, whereby their angle of incidence can be adjusted to be vertical, inclined, or tangential relative to the perforations of the skin, and whereby this angle of incidence can also be reversibly readjusted by means of additional devices.

In a further form of embodiment of the invention, this [device] is used in the form of a patch-like dermal or transdermal non-invasive or micro-invasive diagnosis system in order to improve and expand practical usage, whereby the integrated sensor devices are configured in the form of planar electronic chemosensors.

In a further form of embodiment of the invention, this [device] is used in the form of a patch-like dermal or transdermal non-invasive or micro-invasive diagnosis system in order to improve and expand practical usage, whereby the sensor device can be pushed into, or removed from, it [the diagnostic system] in a reversible manner.

In a further form of embodiment of the invention, this [device] is used in the form of a patch-like dermal or transdermal non-invasive or micro-invasive diagnosis system in order to improve and expand practical usage, whereby the analysis of the fluid takes place by means of a chemical test strip that can be pushed into, or removed from, it [the diagnostic system] in a reversible manner.

In a further form of embodiment of the invention, this [device] is also used in the veterinary sector in order to improve and expand practical usage.

The advantages of the invention arise, in particular, as a result of the feature that the patch-like chip systems that have been described permit programmed and “intelligently” controlled effects, and they are hereby capable of being used in basically two directions. On the one hand, [these effects are usable] by way of the feature that the heat that is conductively emitted by them is used directly in the form of a final biological effect and, on the other hand, [these effects are usable] via a mechanism that can be termed indirect thermodynamic intensification.

An indirect thermodynamic effect in the therapy sector is, for example, a controlled relaxation effect via the skin, e.g. in cases where neuromuscularly engendered spasms or neurological metabolic diseases are present. This is therefore a primary physical therapeutic effect in which an exogenous pharmacological agent, which mediates the effect, is unnecessary. An additional application, which is likewise direct though diagnostic, is, for example, the activation, which is induced via locally controlled hyperthermia, and the collection of fluids from the skin as a result of intensified perspiration and intradermal hydration, sweat, and interstitial dermal fluid as well as their direct analysis by means of integrated micro-sensors, e.g. via electronic chemosensors, ion-selective probes, and also chemical test strips. Depending on the layer, this permits a non-invasive or micro-invasive diagnosis by substances that are inherent to the body, such as electrolytes and glucose, and also that by pharmacologically active extraneous substances, e.g. alcohol. medicinal preparations, and also drugs. It is especially within the sector of blood glucose determinations in cases of diabetic persons that a non-invasive or merely micro-invasive measurement is very advantageous since the current procedure for these patients is painful and tiresome and, in addition, it involves numerous sources of error. Since a micro-invasive method via the interstitial dermal fluid as the analyte also plays a role in the case of the low-nociceptor and also essentially vessel-free epidermis, such a determination is pain-free and blood-free here as well. Since the epidermal interstitial fluid correlates directly with the blood values as well (Bantle J. P., Thomas W.: J. Lab. Clin. Med. 130 (1997) 436-441), it also permits comparable accuracy to that in the case of capillary blood even though it makes use of very small volumes (Service F. J., O'Brien P. C., et al.: Diabetes care 20 (1997) 1426-1429).

The second direction of application is the indirect exploitation of thermodynamics as a secondary effect, i.e. as a diffusion intensifier for dermal or transdermal release systems. In this regard, the transdermal release systems here can either be solid mechanical devices, e.g. passive transdermal patch systems, or semi-solid pharmaceutical formulations, e.g. skin ointments that contain an active substance. Deposits of substances that are located beneath the surface of the skin, e.g. slow release suspensions of crystals, or colloidally dispersed formulations, or deposit formulations comprising biologically compatible substances with analgesics or with hormones, such as e.g. insulin, are also dermal release systems. In the case of these, the disintegration of these otherwise very slowly soluble deposits can then be increased thermodynamically in a transcutaneous manner; as a result of this, an acutely increased release takes place of the substances, which are contained in them, into the blood circulation system. Thus, in overall terms, more intense release and also intensified resorption can be produced from different dermal or transdermal pharmaceutical formulations as a result of these secondary effects, whereby such release and resorption is controlled in a temporal thermodynamic manner, or even in a dose-dependent manner either as a response to acute demand or in a pre-programmed manner as well.

As additional advantages of the invention, the feature is present that the patch-like chip systems that have been described are now also capable of controlling and regulating, in an individually adapted manner, the effects of the previous purely passive systems. As a result of the interactive regulating and controlling components that are integrated within the system, a controlled influence is exerted on the thermodynamic activities of the coupled release system. This can take place in a reproducible manner over extended and defined periods of time, and in defined doses, and also in the form of a response to a direct demand by the user.

The basic physical system for the patch-like chip systems for such complex usage can also be described mathematically, in overall terms, as the indirect partial activation of the Bateman function—i.e. the time/concentration curve (that arises in the form of the pharmacokinetic resultants of the invasion and evasion processes) of substances in the blood, i.e. as a consequence of the activation of the diffusion conditions. Such activation takes place in this regard via the chip-controlled conductive transfer of heat, which is derived from the Fourier law of thermal conduction, as a result of which patch-like systems, which operate in the form of quasi “Fourier systems”, therefore produce a controlled “emissive power”.

Transdermal diffusion hereby increases in proportion to the increase in local temperature, i.e. in accordance with Fick's law dQ/dt=DF(C₁−C₂/d). Conductive intensification follows Fourier's law for the conductive transfer of heat dQ/dt=−λA(dT/dx). This determines the heat flow factor Q for a given temperature profile T and proportionality rate [sic; constant?] of the material that is used (the thermal conductivity λ). The rate of heat flow dQ/dt through a homogeneous solid is proportional to the surface area A, i.e. to that part of the surface that is perpendicular to the direction of the flow of heat, and to the temperature difference along the path of the flow of heat, i.e. dT/dx. This in turn leads, via intensification of the diffusion parameters, to changes in the absorption parameters of the Bateman function: C=[illegible equation; page 14, line 16], whereby in this connection: C₀=hypothetical initial concentration; k_{[illeg]}=a constant relating to the rate of invasion; k_{[illeg]}=a constant relating to the rate of elimination. The overall combination of the aforementioned interdependent factors is therefore:

dQ/dt=−λAdT/dx [symbol] dQ/dt=DF(C₁−C₂/d) [symbol] C=[illegible equation; page 14, lin

The possibility is hereby opened up, as an additional advantage, namely that of computationally estimating the biological effects that can be expected thermodynamically.

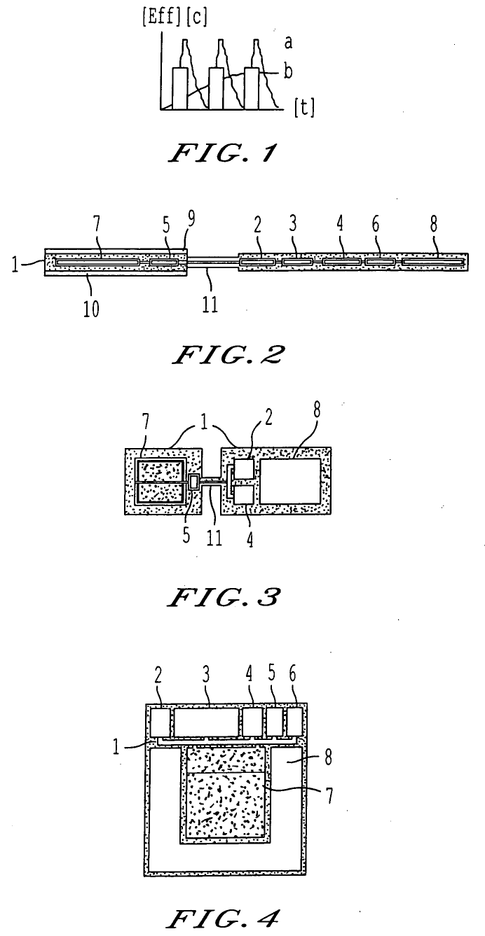

These basic characteristics are illustrated schematically in FIG. 1. In the case of triggering heat pulses (gray columns) that are induced in a defined temporally limited manner, pulse-like increases in substance release occur in the case of a passive transdermal system (curve a), which is thereby thermodynamically activated, and hence an increase in the serum concentration of the substance [C] or an increase in the degree of pronouncement of the effect [Eff] as a function of time [t] arises. After thermodynamic activation has ended, the serum curve, and hence the degree of pronouncement of the effect, decline once again. In contrast to this, a purely diffusion dependent, passive system (curve b), without thermodynamic activation, merely releases the active substances that are contained therein in accordance with the existing concentration differences, and it therefore merely follows the characteristics of continuous 1st order invasion kinetics.

Additional advantages of the invention are that, depending on the requirement and usage objective, the control elements of the chip systems can be configured either as demand-based systems, e.g. in a mode that has been programmed in a fixed manner with selection possibilities (open-loop), or in the form of feed-back systems that are linked via individual or multiple integrated sensors (closed-loop) with various program options. Various applications are permitted in this regard depending on the scope of the specific programming of the microprocessor. Thus, for example, the consecutive release kinetics can be adapted to both the physiological and the individual conditions and requirements via time/heat profiles that are preprogrammed in a free or fixed manner. Permeations can then be pulsed, e.g. either at defined intervals of time or at such times of the day that they follow in an improved manner the circumstances that are involved in the so-called circadian rhythms. Adaptive counter-reactions can also be reduced in this way, e.g. in the case of substances that exhibit tolerance phenomena, such as nitroglycerine. Thus, as is shown schematically in FIG. 1, demand-based acute individual dose adaptation is also possible, e.g. in the case of a clinically rapidly required higher dose of an analgesic, whereby this is not possible with passive systems. As a result of incorporating remote control elements (remote control) into the patch-like chip system, e.g. via infrared, a change in the dose for a patient can, if required, also be initiated electronically by the doctor who is providing treatment or by the nursing staff. In an enlargement of these operational options, the treatment procedures in question can also be stored in the microprocessor, and then they can be read off in a wireless manner via computer interfaces and they can be processed further and documented in a computer.

These individualized adaptations of the medicament dose hereby increase both the quality of life of the patient and also the safety of the therapy by reducing the undesired effects of the medicament. Suitable applications are, for example, pain therapy or therapy in the area of central mood disorders. In contrast to transdermal systems that operate iontophoretically, the skin does not come into contact either with electrical parts or with electric currents in the case of a thermodynamically activated system. Safety during usage and local tolerance by the skin are thus distinctly greater than for iontophoretic systems. In addition, the breadth of application is greater since iontophoretic systems are capable of operating only with ionizable substances. The required local temperature differences for the thermodynamic activation of coupled systems amount to only a few degrees Celsius, and they are therefore innocuous both locally for the skin and for the entire organism as well.

Since the patch-like chip systems operate in the form of an integrated and interactively controlled thermodynamic activator, a principal sector in the area of therapeutic transdermal applications is also their coupling to pre-existing and clinically applied passive transdermal therapies. Thus, as a result of coupling, they can also optimize pre-existing therapies by opening these up to improved regulation possibilities and individual control. Thus the triggering of such transdermal reactions, which are now controlled in an “intelligent” manner, shows significant medical advantages relative to the effects of purely passive transdermal systems.

A technical advantage is the fact that the patch-like chip systems can be manufactured with production devices that have already become conventional, namely in large numbers, and in a profitable manner, and in an exactly standardized and reproducible way, and also the fact that they can be variably provided with usage based dimensions. As a result of their novel and, in overall terms, flexible patch-like configuration, and incorporation into flexible materials, and the degree of pronouncement of mechanically flexible components, the patch-like chip systems also permit roll-to-roll production templates in the way in which these are also used in e.g. printing techniques or in sub-process in microelectronics. This is not possible with otherwise conventional fixed techniques with their rigid supporting components. In addition, this also permits and simplifies their incorporation into pre-existing pharmaceutical roll-to-roll production systems, such as are used e.g. in the case of transdermal patches, and in the case of bandages. In this way, the patch-like chip systems can also be adapted to, and fixed to, existing dermal or transdermal systems in a complementary manner in regard to dimensions. In addition, they can also be tested in an automated manner in terms of their functional capability using the roll-to-roll process.

Basic technical examples of the invention are explained below though without wanting to restrict the invention technically to these examples.

FIG. 2, in the form of a schematic cross section, shows the basic structure of a patch-like chip system in which functionally different parts of the communal flexible supporting matrix are configured in sectors that are separated two-dimensionally. In this case, the following components are located in a communal supporting matrix (1) that comprises a flexible polymer: an externally accessible switch (2) for activating the system; optionally a display (3), e.g. one comprising light emitting diodes; a microprocessor (4), which is equipped with various connection options, in the form of a central controller and optionally also a specific operational sensor device (5) for one or more sensors, e.g. for temperature or humidity control, or for determining specific substance concentrations; and optionally a transmitting and receiving station for wireless remote operation (6), e.g. an interface for triggering via infrared. Discrete parts, such as capacitors and resistors, have not been itemized in this arrangement. Components 2-6 are hereby connected directly and interactively to a device (7), namely the thermodynamic actor, that produces heat electrically. For example, this can be a flexible printed resistance circuit or even a continuous thin carbon layer that has been applied to a flexible foil. The structure is also connected to an energy source (8) that comprises e.g. a mechanically flexibly configured ultra-flat lithium/polymer battery.

For the purpose of limiting its dimensions in terms of height, the microprocessor in this case has been embedded in the flexible matrix, i.e. it has been installed without an insulating layer and thus in the form of a “naked” processor structure, and it can also be mechanically elasticized by means of additional operational procedures in order to reduce its layer thickness. The battery volume is distributed two-dimensionally as a result of the specific design. In this example, a thin covering layer with a heat reflecting lining (9) is also positioned on the upper side of the supporting matrix, whereby this covering layer unilaterally reduces thermal irradiation that is directed upward. A thermo-resistant adhesive layer (10) is located on the underside of the matrix. This [adhesive layer] serves for reversibly fixing the patch-like chip system to a mechanical surface, e.g. to a transdermal patch-system to which the chip system is coupled, or it even serves for reversible fixing to the skin, e.g. in the case of its usage as a dermal system. The matrix part with the thermodynamic actor and control panel are constructed in a separated manner by means of a thin matrix bridge (11). This type of “tender device” increases the possibility of flexible applications, e.g. those that are connected via torsion. The total thickness of such types of flexible patch-like chip system is usually distinctly less than 1 mm, and the overall height of such devices can be between 10 μm and 2,000 μm. The actor is capable of producing a controlled regional temperature increase at pre-selected intervals of time in the area of the surface of the skin that is located beneath it, e.g. during application to the skin, by optionally between 1° C. and 6° C., whereby this corresponds to absolute temperature ranges between approximately 36° C. and 42° C. Temporally more extended temperatures above 42° C. are generally injurious to the skin.

FIG. 3 shows the device of FIG. 2 in the form of a schematic plan view, whereby here, however, the incorporation of a display and a remote control unit has been omitted. As in FIG. 2, the thermodynamic actor (7) is connected to an additional part, which supports the switch (2), the microprocessor (4), and the energy supply (8), whereby such a connection is effected in an electrically conductive manner via an operational sensor (5) and in a flexible manner via a cross-piece (11), which comprises the material of the supporting matrix, whereby this connection (11) can also optionally take place by means of a reversible electrical coupling arrangement, e.g. by means of a plug-type or magnetic coupling arrangement, that is built in there. Additional segmentation of the control panel matrix is technically possible. An additional increase in spatial flexibility and variability arises as a result of this form of configuration, such as e.g. in the case of surfaces of differing topography, and also the ability to exchange sensors and/or energy sources in the case of differing requirements.

FIG. 4, in contrast to FIGS. 2 and 3, shows a fully integrated system in the form of a schematic plan view, whereby all the components are arranged in a direct spatially coherent manner in the flexible matrix (1), i.e. the actor (7) and the complete control panel with its different regulating and control components (2-6). In this design, the thermodynamic actor (7) is installed in the matrix in a technically centered manner. The battery (8), which serves as a source of energy, has been spread out around the entire actor for the purpose, on the one hand, of reducing its dimensions in terms of height and, on the other hand, for the purpose of increasing its flexibility two-dimensionally and in a U-shaped manner.

FIG. 5, in the form of an exploded arrangement, likewise shows a fully integrated system for the purpose of therapeutic usage in which all the components are arranged in a direct spatially coherent manner. In the case of this patch-like chip system, the adhesive layer of the chip system (12) is configured in a circular manner, whereby this is within the framework of a passive transdermal system (13) that is to be coupled to it. This system is thus adhesively attached in a circular manner around the area of a transdermal system that is already located on the skin. If the therapeutic transdermal system comprises a semi-solid pharmaceutical formulation, e.g. a gel that contains an active substance, then such a configuration has the advantage that the system could not be mechanically fixed to such a semi-solid layer. Thus, in this case, there is no direct mechanical connection between the patch-like chip system and the transdermal system. If required, however, the same technical design can be used in the case of a solid patch system if, likewise, no mechanical connection is required to take place there. The active substance matrix, which is located in the coupled transdermal system (13), is then conductively thermodynamically activated via the actor (7) at the time of triggering the program, which is contained in the microprocessor (4), via the switch (2). In contrast to the previous purely passive diffusion rate, the release of the active substance is increased in the coupled transdermal system by a pre-programmed thermodynamic activity factor.

FIG. 6, in the form of a schematic plan view, shows the basic design of a patch-like chip system for the physically direct therapeutic application of local hyperthermia, e.g. in cases of pain that is caused neuromuscularly. In this case, the matrix (1) with the actor (7), which is contained therein, is applied directly and adhesively to a medicinal patch (14). Here, the actor is connected in an electrically conductive manner to an additional flexible matrix component via a matrix bridge (11). This second component contains the microprocessor (5) that, for its part, is connected in a conductive manner to a plug-type connection (15). Electrical energy, for example, can be supplied to the system via this plug-type connection with use being made of a flexible line.

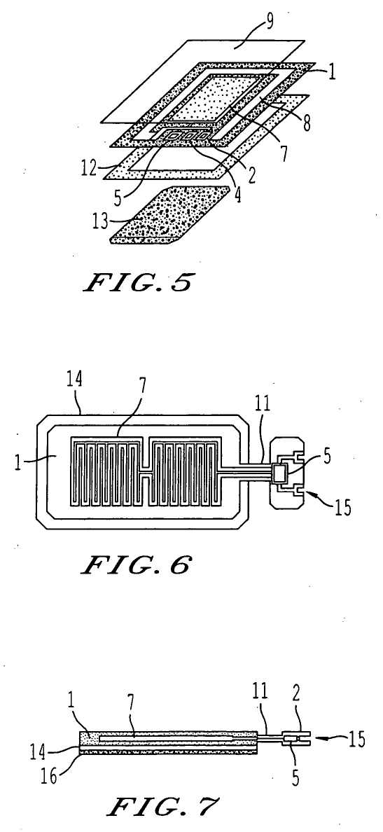

FIG. 7, in the form of a schematic cross section, shows the same structure as FIG. 6. In this case, the layer for adhesion to the skin is also itemized, whereby this layer is located below the medicinal patch (14).

FIG. 8, in the form of a schematic cross section, shows a modification of the basic structure of a dermal diagnostic system. In this technical example, one is dealing with the collection of fluid from the skin, whereby this fluid emerges onto the surface of the skin via thermodynamically induced hydration, together with its quantitative or qualitative analysis by means of an integrated micro-sensor device, e.g. an electronic chemosensor or even a chemical test strip. Here, the thermodynamic actor (7), which is located in the flexible supporting matrix (1), is designed in a circular manner around a planar sensor device (17) that is integrated into the matrix. The fluid that has emerged onto the surface of the skin is absorbed cohesively by a plate-like device with one or more capillary channels (18), and then it is led to the surface thereof that is directly opposite the measurement area of the sensor. The surface of the device hereby topographically forms a component of an integrated micro-chamber that is also connected to one or more ventilation channels (19). The sensor device, for its part, is connected to a specific operational sensor processor (5) and to the microprocessor (4). The system is also applied to a medicinal supporting patch (14) that is provided on its underside with a layer (16) for adhesion to the skin, and it contains perforations toward the skin in the area of the capillary device (18). Applications are e.g. painless and blood-free non-invasive patch systems for the analysis of substances that emerge onto the surface of the skin from the circulation system via organs, which are supplementary to the skin, and/or interstitially or in a transcellular manner, and are technically detectable with the help of chemosensors, microprobes, or test strips, e.g. electrolytes, adrenalin, glucose, lactate, certain medicinal preparations, alcohol, or certain drugs. The evaluation of the findings can take place via a PC interface of the microprocessor, whereby this evaluation can first be read off in the PC, and then it can be processed and documented there.

FIG. 9, in the form of a schematic cross section, shows an additional modification of the basic structure of the patch-like chip system for dermal diagnostic purposes. One is dealing here with a micro-invasive quantitative or qualitative analysis of the interstitial fluid (ISF) from the epidermal layer of the skin by means of a specific planar micro-sensor (17) that is integrated into the matrix (1). The hydration of this layer of skin is intensified thermodynamically via the actor (7) in addition to the hydration that has already been intensified by the patch engendered mechanical occlusion of the surface of the skin. This [actor] is applied in a circular manner around the sensor. The ISF from the epidermis is absorbed by means of a plate-like device with one or more micro-tubes (20), which are, for their part, immersed in the ISF from the epidermal layer of skin and, in part, it is cohesively sucked up and led to the surface thereof that is located opposite the measurement area of the sensor. However, an important physical mechanism in this connection is the temperature engendered increase in the subcutaneous flow of blood with consecutively increased hydration of the epidermal cellular and intercellular distribution zone. Since this induced regional hyperthermia also increases, inter alia, the interstitial circulation of the epidermal ISF, a type of circulation pump mechanism arises so that the absorption and forwarding of the epidermal fluid is promoted as a result of regionally increased hydrostatic pressure in accordance with the principle of an artesian well. The surface of the absorption device topographically forms a component of an integrated micro-chamber that is additionally connected to one or more ventilation channels (19). As described in embodiment example 8, this modification is also applied to a medicinal supporting patch (14) that is provided on its underside with a layer (16) for adhesion to the skin, and the patch area is perforated toward the skin around the epidermal micro-tubes (20).

In the case of a specific glucose determination, the chemical reaction can take place, for example, with the help of the glucose oxidize enzyme that is integrated into the sensor, and the reaction potential is hereby depicted amperometrically, for example.

In the case of a modification of the same system with use being made of a non-planar sensor in which the [micro-]tubes have already been doped with the glucose oxidize enzyme, the fluid can even be analyzed epidermally in an in situ manner as well, whereby this then opens up the possibility of continuous in situ measurement as well. Since the induced regional hyperthermia also increases the interstitial circulation of the epidermal ISF, a gradient for the sensor can be maintained permanently there in accordance with a sort of circulation pump mechanism.

The very thin upper epidermal layers are largely free from blood vessels and terminal pain receptors, and they therefore require micro-invasive distances of only approximately 1-1.5 mm for the micro-tubes, whereby only the uppermost, very thin keratin layer of the skin, i.e. the stratum corneum, has to be penetrated. Suitable applications of this technical example are therefore painless and blood-free micro-invasive dermal diagnostic patch systems for the analysis of substances that emerge into the interstitial fluid of the epidermal skin layers from the circulation system, and they are technically detectable with e.g. electronic chemosensors or even chemical test strips. Glucose, lactate, electrolytes, adrenalin, creatinine, certain medicinal preparations, and also certain drugs belong to this [group of substances]. Analysis and evaluation take place as described in embodiment example 8. The evaluation and documentation of the findings can take place via the PC interface of the microprocessor.

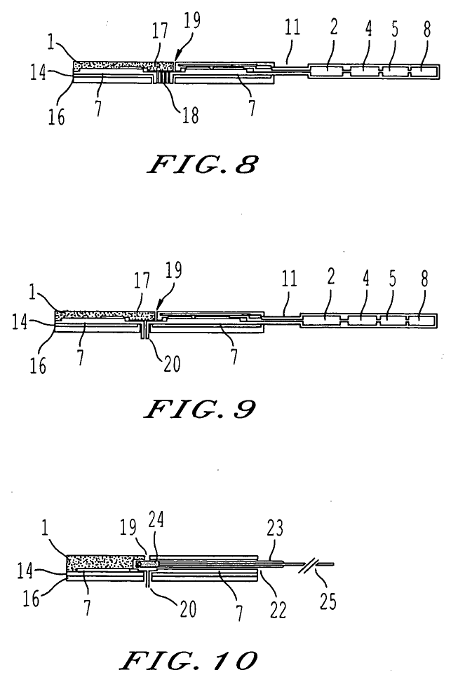

FIG. 10, in the form of a schematic cross section, shows a further modification of the basic structure of the patch-like chip system for dermal diagnostic purposes. One is dealing here with a micro-invasive variant for the analysis of the interstitial fluid (ISF) from the epidermal layer of the skin as has already been illustrated in FIG. 9. In this modification, however, a pre-manufactured, slot-like guidance device (22) is located in the supporting matrix of the system, whereby either an electronic sensor device, which is constructed in a planar manner, or a conventional chemical test strip (23) can be introduced reversibly into the system via this guidance device. In the inserted state, the read-out window of the sensor or the reaction zone of the test strip (24) is positioned directly above the ISF that has emerged and is in contact with it. In the case of an electronic sensor, this is then electrically connected to the evaluating control panel via a communal measurement and supply line [assumed typo] (25). This modification therefore permits the repeated use of the same sensor with different patch systems via the same basic principle. In the case of a chemical test strip, the patch also contains an electrical contact for connection to the electrical supply system for the thermodynamic actor.

In accordance with FIG. 8, a similar planar construction with reversible sensor or test strip usage can also, naturally, be used for the non-invasive system for the analysis of fluids from the surface of the skin.

1. Flexible chip systems for the thermodynamic activation and control of dermal and transdermal systems, characterized by the feature that use is made of patch-like chip systems for the thermodynamic control of topical dermal and transdermal systems, whereby these are composed in the form of a multi-component system that is configured in a patch-like manner in such a way that they comprise a source of electrical energy, which is located in a communal supporting matrix, and a programmable microprocessor, which serves as a thermo-controller, and an activation circuit, whereby these are, for their part, technically connected to a device that produces electrically induced heat, and whereby the patch-like chip system can, in overall terms, be applied in a complementary manner to a topical dermal or transdermal system in such a way that the heat profile that is produced is transferred to the topical dermal or transdermal systems in such a way that these [systems] are thermodynamically activated in a controlled form.

2. Devices in accordance with claim 1, characterized by the feature that the communal supporting matrix is geometrically subdivided into operational function sectors that are mutually connected in an electrically conducting manner, whereby the connections between these function sectors can be configured in a reversible manner.

3. Devices in accordance with the preceding claims, characterized by the feature that the matrices and technically active components of the patch-like chip systems are composed of certain materials that possess mechanically elastic or plastic properties, and that are optically transparent or opaque, and that possess electrically conductive or magnetic properties, and that, chemically, are non-metallic polymers of natural or synthetic origin or they are metallic materials.

4. Devices in accordance with the preceding claims, characterized by the feature that additional electrical, electronic, magnetic, micro-mechanical, chemical, or chemo-technical components or combinations thereof are incorporated into these devices for specific usage purposes.

5. Devices in accordance with the preceding claims, characterized by the feature that control of the induced heat profile takes place either using an open-loop control technique or a closed-loop technique with feed-back via sensors.

6. Devices in accordance with the preceding claims, characterized by the feature that these contain devices for the reception and transmission of remote control signals, whereby such reception and transmission can take place either physically (via infrared, ultrasound, electromagnetic waves, or laser techniques) or in a chemosensory manner via chemically volatile substances.

7. Devices in accordance with the preceding claims, characterized by the feature that the thermodynamic actor can also be triggered in sub-surfaces, including those with different temperatures.

8. Devices in accordance with the preceding claims, [characterized by the feature] that the thermodynamic actor can be configured in the form of all possible two-dimensional geometries.

9. Devices in accordance with the preceding claims, [characterized by the feature] that their production takes place technically, in parts or wholly, using roll-to-roll processes.

10. Devices in accordance with the preceding claims, characterized by the feature that these devices are used therapeutically in dermal and transdermal systems that do not contain pharmacologically active substances.

11. Devices in accordance with the preceding claims, characterized by the feature that these devices are used therapeutically for the purpose of regional hyperthermia for locally heating tumor cells, especially those in cases of tumors in the breast region, the skin region, or the genital region.

12. Devices in accordance with the preceding claims, characterized by the feature that these devices are used therapeutically in topical dermal or transdermal systems that contain the following as pharmacologically active substances: nitroglycerine, fentanyl, sufentanil, buprenorphine, morphine, hydromorphine [sic; hydromorphone?], lidocaine, indomethacin, ibuprofen, diclofenac, piroxicam, nicotine, clonidine, estradiol, progesterone, testosterone, norethisterone, oxybutynin, buspirone, scopolamine, including their chemical analogs, derivatives, isomers, and salts, either in the form of individual substances or in the form of combinations.

13. Devices in accordance with the preceding claims, characterized by the feature that these devices are used therapeutically in dermal or transdermal systems that comprise semi-solid or fluid forms as the pharmaceutical formulation, especially ointments, gels, creams, lotions, emulsions, suspensions, or solutions.

14. Devices in accordance with the preceding claims, characterized by the feature that these devices are used for the accelerated disintegration of epidermal or dermal deposits of active substances, especially deposits containing the following hormones: insulin, growth hormone, estradiol, progesterone, testosterone, including their chemical analogs.

15. Devices in accordance with the preceding claims, characterized by the feature that these devices are used in the form of patch-like dermal or transdermal diagnosis systems for collecting and analyzing the natural fluid from the skin, sweat, and the interstitial dermal fluid, and especially for analyzing the following substances that are contained therein: glucose, lactate, electrolytes, adrenalin, creatine, alcohol, along with medicinal preparations and drugs.

16. Devices in accordance with the preceding claims, characterized by the feature that these devices are used in the form of patch-like dermal or transdermal non-invasive diagnosis systems, whereby the collection and analysis of the fluid, which emerges onto the surface of the skin, takes place by means of collection and sensor devices, which are integrated therein, and whereby the thermodynamic actor is arranged around them in a circular manner, and whereby the fluid from the skin is absorbed by a plate-like collection device, which is equipped with capillary channels, and the fluid is analyzed and evaluated by means of electronic chemosensors or chemical test strips, which are in contact with the fluid, and whereby this is used for the non-invasive analysis of, in particular, glucose, lactate, electrolytes, adrenalin, creatine, medicinal preparations, alcohol, and drugs.

17. Devices in accordance with the preceding claims, characterized by the feature that these devices are used in the form of patch-like dermal or transdermal micro-invasive diagnosis systems, whereby the collection and analysis of the interstitial fluid from the skin takes place by means of an integrated collection and sensor device, and whereby the thermodynamic actor is arranged around it in a circular manner, and whereby the interstitial fluid from the skin is absorbed or contacted by a plate-like collection device, which is equipped with micro-tubes, and whereby this collection device is suitable for penetrating the uppermost epidermal layer of skin, and the fluid is analyzed and evaluated by means of electronic chemosensors or chemical test strips, which are in contact with the fluid, and whereby this is used for the micro-invasive analysis of, in particular, glucose, lactate, electrolytes, adrenalin, creatine, medicinal preparations, and drugs.

18. Devices in accordance with the preceding claims, characterized by the feature that these devices are used in the form of patch-like dermal or transdermal non-invasive diagnosis systems, whereby the collection and conveying device for the fluid from the skin comprises, wholly or in parts, hollow polymeric fibers, micro-tubes, or hollow probes, which are made from a metallic, polymeric, or ceramic material, and whereby their angle of incidence can be adjusted to be vertical, inclined, or tangential relative to the perforations of the skin, and whereby this angle of incidence can also be reversibly readjusted by means of additional devices.

19. Devices in accordance with the preceding claims, characterized by the feature that these devices are used in the form of patch-like dermal or transdermal non-invasive or micro-invasive diagnosis systems, whereby the integrated sensor devices are configured in the form of planar electronic chemosensors.

20. Devices in accordance with the preceding claims, characterized by the feature that these devices are used in the form of patch-like dermal or transdermal non-invasive or micro-invasive diagnosis systems, whereby the sensor devices can be pushed into them, or removed from them, in a reversible manner.

21. Devices in accordance with the preceding claims, characterized by the feature that these devices are used in the form of patch-like dermal or transdermal non-invasive or micro-invasive diagnosis systems, whereby the analysis of the fluid takes place by means of chemical test strips that can be pushed into them, or removed from them, in a reversible manner.

22. Devices in accordance with the preceding claims, characterized by the feature that these devices are also used in the veterinary sector.
